(19) United States
(12) Patent Application Publication
(10) Pub. No.: US 2017/0014793 A1
(43) Pub. Date: Jan. 19, 2017

(54) Single Use, Low Dead Volume Apparatus For Liposome Sizing or Extrusion

(71) Applicants: Graham Jeffrey Taylor, (US); Nima Tamaddoni Jahromi, (US)

(72) Inventors: Graham Jeffrey Taylor, Knoxville, TN (US); Nima Tamaddoni Jahromi, Knoxville, TN (US)

(21) Appl. No.: 15/209,193

(22) Filed: Jul. 13, 2016

Related U.S. Application Data
(60) Provisional application No. 62/191,927, filed on Jul. 13, 2015.

(51) Int. Cl. B01J 13/04; A61K 9/127

(52) U.S. Cl. CPC B01J 13/04; A61K 9/1277

(57) ABSTRACT

A single-use, extrusion apparatus and method of using the apparatus. The apparatus includes a first housing member having a first port dimensioned to receive a first needle-tipped syringe, and a first needle path dimensioned to receive a first needle, wherein the first needle path extends from the first port through the first housing member; a second housing member having a second port dimensioned to receive a second needle-tipped syringe, and a second needle path, dimensioned to receive a second needle, wherein the second needle path extends from the second port through the second housing member and is collinear with the first needle path extending through the first housing member; and a membrane filter disposed between the first and second housing members, wherein a total dead-volume of the apparatus, not including the first and second needle paths is less than or equal to about 50 microliters.

### CROSS REFERENCE TO RELATED APPLICATIONS

The present application claims priority to U.S. Provisional Patent Application No. 62/191,927, filed Jul. 13, 2015, which is hereby fully and completely incorporated by reference herein.

### FIELD OF THE INVENTION

The present disclosure relates to a single-use, fully assembled, disposable syringe filter apparatus, such apparatus being applicable to the extrusion or sizing of liposomes using low volumes of either aqueous or non-polar solutions, such solutions also containing lipids, cholesterol, and/or other components of cell membranes, or synthetic or naturally derived variants or artificially constructed mimics of such components, which components are desirable to be extruded or sized for research or commercial purposes. The present disclosure further relates to the methods of making and using the aforementioned apparatus.

### BACKGROUND

Lipids are the building blocks of cell membranes, and scientists and engineers are continually finding new applications for lipids and lipid-based constructs, such as drug delivery and gene transfection, and in scientific studies as materials for constructing model lipid bilayers (planar bilayers, giant unilamellar vesicles, large and small unilamellar vesicles) and other lipid-based constructs, nanodiscs, and nanocarriers. Other lipid-like molecules, membrane components, and even synthetic molecules may be used in the creation of vesicles and self-assembled aggregates in solution; such molecules may be charged or uncharged and include lipids as well as cholesterol, signaling lipids, lipid A, cardiolipin, proteins, surfactants, and amphiphilic polymers.

Often, lipids or similar amphiphiles are dissolved in an aqueous medium, such as buffered water, and can be further processed to produce unilamellar vesicles (hereafter referred to as “UV” or “liposome”). When the lipids or amphiphiles are initially dissolved, their amphiphilic structure drives self-assembly into sheets such that the hydrophobic lipid tails are not exposed to the surrounding water medium. Most commonly, the resulting structures are multilamellar vesicles (hereafter referred to as “MLV”), which may be layered onion-like spheres or vesicles, wherein each layer may comprise self-assembled vesicles of lipids or amphiphiles. For many applications, the MLV may be further extruded through a membrane having pores of specific diameter (typically about 100-500 nm in diameter), or may be processed by sonication via a tip or bath style sonicator that delivers high energy sound waves that break up the MLV into smaller UV. The present disclosure is specifically related to the art of small volume MLV extrusion.

Several devices exist that can be used for such liposome extrusion, and these devices can be classified into two main categories: those intended for extrusion of large volumes (for example, volumes greater than or equal to 10 mL) and those intended for extrusion of smaller volumes (for example volumes of less than 10 mL). Existing small-volume extrusion options include luer-connected syringe filters (Pall Corporation, Whatman, Cole-Parmer, Millex, etc), the MiniExtruder (Avanti Polar Lipids, Inc.), the LiposoFast (Avestin), or the Lipex Extruder (Northern Lipids, Inc.).

The existing small-volume extrusion devices all share several disadvantages. General syringe filters are often permanently bonded, and may often be provided free of contamination on a single-use basis, however these types of filters are intended for use simply as a filter, such as to remove particulate or materials from the fluid being passed through the filter. Accordingly, these sorts of filters are not suitable for liposome extrusion since the pore size of syringe filters is generally greater than 1000 nanometers (nm) and is typically about 1000 nm and the dead volume of the syringe filters is greater than 0.5 mL.

A further disadvantage of syringe filters is the fact that there are no known or available syringe filters designed to accept needle-tipped syringes, and thus fail to provide a significant reduction in the dead-volume, or the amount of solution lost simply due to filling of the filter fluid path. Since small volumes of fluid may commonly be used by researchers performing liposome extrusions, it is extremely desirable to have a single-use extrusion device providing an inherently low dead volume. The dead-volume of general luer-tipped syringe filters often exceed 0.5 mL, which can undesirably cause a greater than 100% loss of the desired extrudant when extruding very small volumes (for example, solution volumes ranging from about 0.1 to about 0.5 mL), and an undesirable loss of extrudant ranging from about 5% to about 50% with small total solution volumes (for example, about 1 mL to about 10 mL). These potential losses are substantial and economically untenable, and thus render general syringe filters unusable for scientists and researchers working with low or small volumes.

There are devices designed specifically for extrusion of large volumes of lipid solution (Lipex Extruder), and in these cases the dead-volume or waste is less significant when compared with the total volume being extruded.

While some known re-usable devices available for extruding small volumes (ranging from less than about 1 mL to about 10 mL) of lipids, liposomes, and other amphiphilic macromolecules do possess low dead-volume fluid paths (Avanti, Avestin), and also accept needle-tipped syringes, such devices require time-consuming cleaning and assembly prior to, and in between, each use.

There is a major disadvantage in the need to clean the pieces of the device before and after each extrusion, as well as the need to manually assemble the device for each extrusion. The need to clean, assemble, extrude, and clean again leads to a significant amount of lost time in every single batch of extrusion. The re-usability of such extrusion devices may also contribute to a significant and undesirable risk of contamination or carryover from sample to sample, despite the required rigorous cleaning procedures.

Furthermore, there can be a significant possibility of operator error when disassembling and/or reassembling the device. Misalignment of the membrane can lead to membrane rupture or leakage, improper assembly or seating of the component parts can lead to leakage and loss of the fluid sample being extruded (which may often be expensive materials). The fact that the samples being extruded may often be highly valuable, and may be comprise specific lipids, proteins, or other costly and sensitive small molecules or macromolecules, indicates that the loss of even a seemingly insignificant amount of solution, such as a fraction of a milliliter, may result in a significant economic loss to the user.

The potential for reducing or eliminating wasted solution when extruding low or small volumes of solutions containing lipids, proteins, amphiphiles, or other biomolecules is a significant factor for desiring a low dead-volume extrusion apparatus. As described above, a significant dead-volume is specifically the reason that general syringe filters (which may often have greater than 0.5 mL dead-volume) are considered unusable by scientists and researchers performing liposome extrusion.

### SUMMARY OF THE DISCLOSED EMBODIMENTS

In one embodiment of the present disclosure, there is provided a low-dead volume liposome extrusion device. The extrusion device may be fully pre-assembled as a clean or sterile single device thereby reducing the risk of contamination from prior extrusions or other sources and improper assembly, and may save valuable time for the user by obviating the need for cleaning and assembly prior to each extrusion. The extrusion device includes a low dead-volume interior fluid path and the device may be used with low dead-volume needle-tipped syringes, or typical luer-tipped syringes through the use of the luer-needle adapter as disclosed herein. Accordingly, the device enables a fluid communication connection between the two syringes and through the disclosed extrusion apparatus, and which furthermore reduces the dead-volume of typical luer-tipped syringes to a low-dead volume as described herein.

The present disclosure provides for a clean, contamination-free, cost-effective single-use extrusion device that can be used without any need for special operator training, and without requiring time for cleaning and assembly prior to, and in between, each use. Thus the assembled extrusion apparatus disclosed herein, including one or more embodiments as shown in the drawings provided herewith, provides significant advantages over previously known devices.

As used herein, the term “dead volume” refers to a volume of fluid or solution, the amount of which solution may be lost, unusable, or not recoverable before, during, or after a procedure due to filling of a filter fluid path with a solution.

Unlike currently commercially available syringe filters and extrusion devices, the presently disclosed apparatus may be constructed so that the entire assembly may be single-use, inexpensive, and disposable, thus ensuring cleanliness and furthermore preventing or reducing possible contamination of the desired extrudant. The disclosed extrusion device may also provide an economic advantage due to the low cost of materials and the reduction in labor time, whereby more extrusion can be done in a shorter period of time, thus lowering the labor cost per extrusion.

As a further economic and utilitarian advantage, the disclosed apparatus may relieve the need for laborious dis-assembly, cleaning and reassembly with each use, thus not only saving valuable time for the user, but further eliminating the possibility of contamination from previous extrusion procedures. Thus, by using the disclosed apparatus and embodiments thereof, a procedure that used to average 30-45 minutes may be done in as little as 2 or 3 minutes, while eliminating operator errors and potential loss of valuable material due to leakage or contamination. The presently disclosed apparatus may also be sterilized, unlike previously known extrusion devices.

As an additional advantage, the presently disclosed apparatus may be configured to reduce the amount of lost or wasted solution via a low dead volume fluid communication path. Furthermore, the apparatus disclosed herein may be designed to accommodate needle-tipped syringes, which may be often used by those skilled in the art due their well known property of inherently low dead-volume.

The presently disclosed apparatus may also be configured to accommodate luer-tipped syringes through the use of a low dead-volume luer-needle adapter that may be included in one or more embodiments of the present disclosure.

### BRIEF DESCRIPTION OF THE DRAWINGS

FIG. 1 is a perspective view, not to scale of a pre-assembled, single-use extrusion apparatus in its ready-to-use state, according to an embodiment of the disclosure.

FIG. 2 is a partial sectional view of the pre-assembled, single use extrusion apparatus of FIG. 1 having a low dead-volume.

FIG. 3A is a cross-sectional view of the pre-assembled, single use extrusion apparatus of FIG. 1.

FIG. 3B is a cross-sectional view of the pre-assembled, single use extrusion apparatus according to another embodiment of the disclosure.

FIGS. 4A and 4B are exploded views of the extrusion apparatus of FIG. 1 showing a filter assembly for the device.

FIG. 5A is a perspective view of one embodiment of a pre-assembled, single-use extrusion apparatus, connected and in fluid communication with low dead-volume needle-tipped syringes for extrusion or sizing of liposomes or other structures in solution.

FIG. 5B is a cross-sectional view of the extrusion apparatus and syringes of FIG. 5A, with syringes and needles inserted into the extrusion apparatus and ready for use.

FIG. 6 is a cross-sectional view of the pre-assembled, single use extrusion apparatus, along with syringes having needles, according to another embodiment of the disclosure.

FIG. 7 is a cross-sectional view of another embodiment of the present disclosure.

FIG. 8 is a graph showing comparative data between the single-use extrusion apparatus disclosed herein, and a reusable extrusion apparatus.

### DETAILED DESCRIPTION

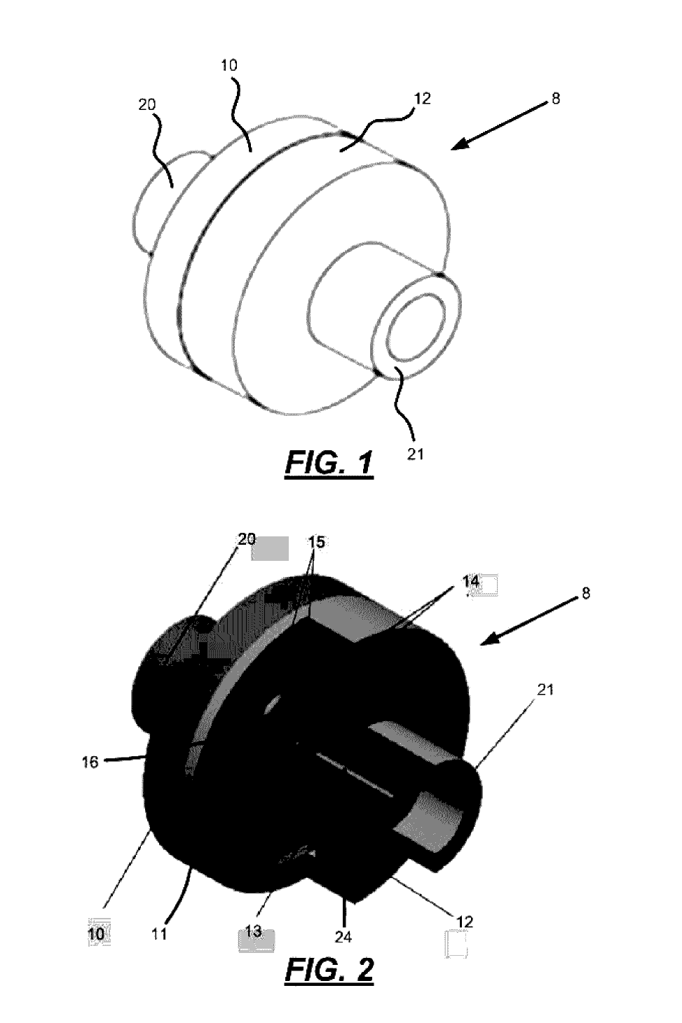

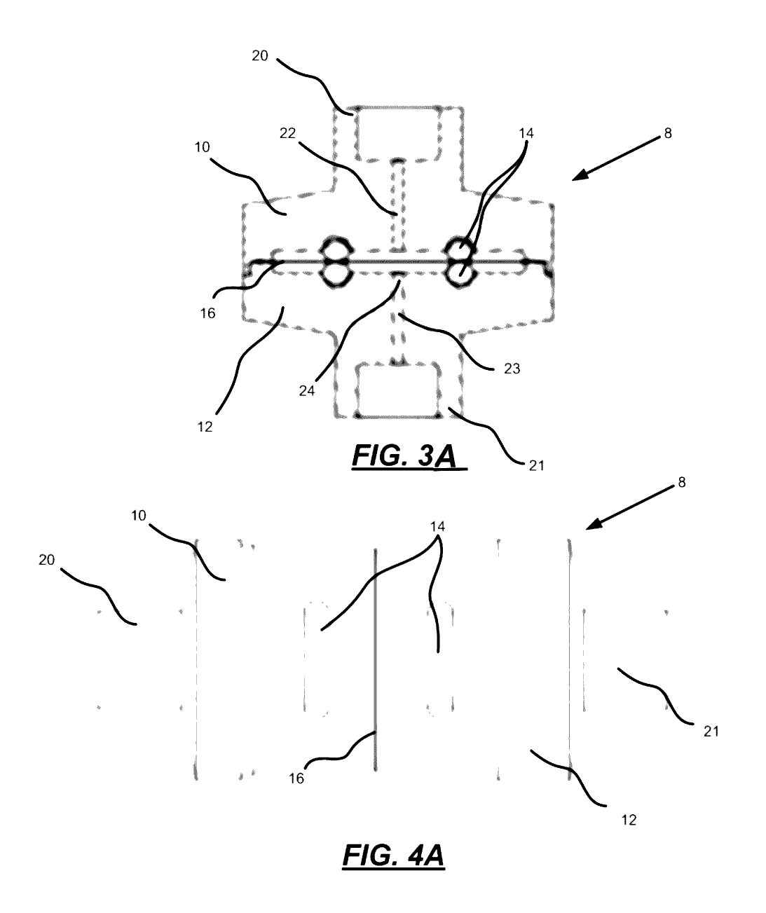

With reference now to FIGS. 1 and 2, the present disclosure provides a fully assembled, single-use, inexpensive, sterile, and syringe-compatible extrusion apparatus 8 that is configured for lipid or liposome extrusion or sizing. The extrusion apparatus 8, includes a first plastic housing member 10, and a second plastic housing member 12 that may be permanently or semi-permanently bonded, connected, or attached so as to create a leak-proof fluid communication path 24 between a first syringe port 20 and a second syringe port 21, through the apparatus 8, the syringe ports being disposed axially on either end of the fluid communication path 24 through the first plastic housing member 10 and the second plastic housing member 12. The first plastic housing member 10 may have an interior face 11, opposite to the first syringe port 20. The second plastic housing member 12 may have an interior face 13, opposite to the second syringe port 21.

The apparatus 8 may be sealed along the peripheral outer edges 15 of each housing member 10 and 12 by chemical or mechanical bonding of the first plastic housing member 10 to the second plastic housing member 12. One or more rubber gaskets, O-rings, or other elastomeric seals 14 may be included in an interior portion of one or more of the housing members 10 and 12 surrounding the fluid communication path 24 through the housing members to provide a leak-proof fluid communication path 24. The leak-proof fluid communication path 24 may be partitioned in the middle by one or more permeable or semipermeable extrusion membranes or filters 16. Accordingly, when the interior face 11 of the first plastic housing member 10 and the interior face 13 of the second plastic housing member 12 are brought together during assembly of the apparatus, the seals 14 may provide mechanical support to the one or more membranes 16 enclosed by the housing members, and may also provide a leak proof fluid communication path 24 in the assembled apparatus 8.

In one embodiment, the housing members 10 and 12 are made of a plastic or thermoplastic material. The housing members 10 and 12 may be generally cylindrical with an overall diameter ranging from about 1 centimeter to about 10 centimeters. Other housing member shapes such as rectangular or cubical may be used provided the shapes are sufficient to reduce the amount of material used to make the housing members 10 and 12.

In one embodiment, each of the housing members 10 and 12 and the fluid communication path 24 through the housing members may be made by injecting a plastic or thermoplastic material into a pre-shaped molding having a removable metal pin, such that when the pin is removed from the housing member, the flow path 24 extends therethrough. Suitable plastics for making the housing members 10 and 12 may include, but are not limited to, polycarbonate, polystyrene, polypropylene, high density polyethylene, and the like. It will be appreciated by those skilled in the art that the housing members may be made from other materials, if economically desirable.

Other embodiments of the present disclosure may include only one rubber gasket or seal 14, or none at all. Other embodiments of the present disclosure may include more than one extrusion membrane or filter 16, for example from 1 to about 20 membranes. In further embodiments, at least one of the interior face 11 of the first plastic housing member 10 and the interior face 13 of the second plastic housing member may provide mechanical support to the membrane 16 in the assembled apparatus 8. The one or more membranes 16 may be a track-etched polycarbonate membrane material having a pore size ranging from about 10 nanometers (nm) to less than about 1000 nm, such as from about 50 nm to about 500 nm and a diameter ranging from about 1 mm to about 15 mm. In other embodiments, the one or more membranes may be anopore inorganic membranes, cellulose acetate membranes, cellulose nitrate membranes, mixed cellulose ester membranes, nylon and polyamide membranes, polyethersulfone membranes, polypropylene membranes, PTFE membranes, regenerated cellulose membranes, track-etched polycarbonate membranes, or any combination thereof.

Bonding or sealing of the first plastic housing member 10 and the second plastic housing member 12 to one another may be accomplished via adhesive, ultrasonic welding, heat based welding or bonding, threaded connection, snap-on or snap-fit connection, chemical or solvent bonding, or other bonding means, including, but not limited to additional design components that may bond or otherwise attach the first plastic housing member 10 to the second plastic housing member 12, so as to maintain a leak-proof fluid communication path 24 axially through the entire assembled apparatus 8.

The assembled apparatus 8 may have a first needle path 22 disposed axially through the first housing member 10. The first needle path 22 may extend from a first syringe port 20 axially through the first housing member 10 to the leak-proof fluid communication path 24. The assembled apparatus 8 may further have a second needle path 23 disposed axially through the second housing member 12. The second needle path 23 may extend from a second syringe port 21 axially through the second housing member 12 to the fluid communication path 24. The first and second needle paths 22 and 23 may be disposed collinearly through the first and second housing members 10 and 12.

The first needle path 22 and the second needle path 23 may be dimensioned to securely and precisely receive needles of various sizes, for example, but not limited to, a 24 gauge needle, or a 22 gauge needle, or more specifically may have a diameter from about 0.01 mm to about 5 mm, and a length substantially equal to, or desirably slightly greater than, the length of the needle for which the apparatus 8 is intended to be used, so that when a syringe having a needle, and containing a fluid, is inserted into a syringe port of either housing member of the assembled apparatus 8, no leakage of fluid may occur as a fluid may be passed back and forth from one side of the membrane 16 to the other. The volume of the entire fluid communication path 24, not including the volume of the first needle path 22 and the second needle path 23, may be less than or equal to about 50 μL.

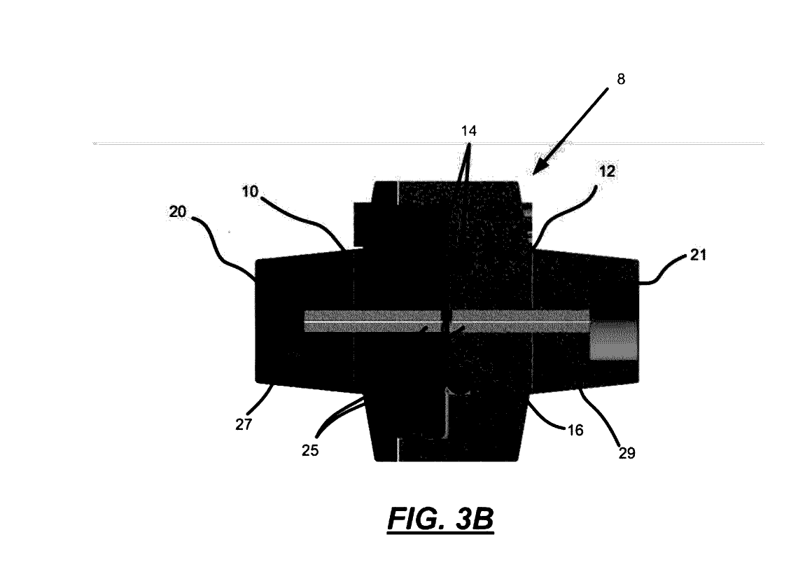

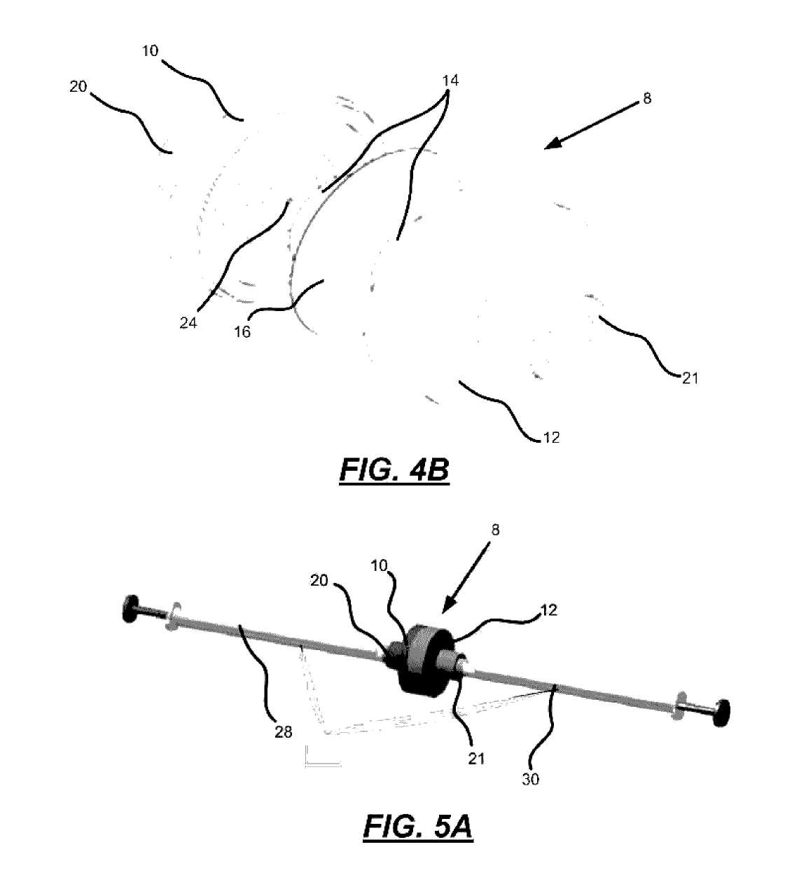

As shown in FIG. 3B, first and second needle paths 27 and 29 may have a tubular insert 25. The tubular insert 25 desirably may have an outer diameter substantially equal to or just smaller than the needle path, and an inner diameter substantially equal to or less than the diameter of the needle for which the apparatus 8 is intended to be used, in order to achieve a tight and leak-proof fit for the needle, and further may be substantially equal in length to the needle paths 27 and 29. The tubular insert 25 may aid in maintaining a leak proof fluid communication connection through the apparatus 8. The tubular insert 25 may be made from a flexible elastomeric tubing, such as surgical tubing or silicone tubing. The tubular insert 25 may also be made from a more rigid material, such as polytetrafluoroethylene, non-protein or lipid binding metals such as stainless steel or titanium, or any suitable curable plastic, thermoplastic, polymer, or copolymer that may be non-protein or non-lipid binding, as will be recognized by those of skill in the art. The tubular insert 25 may be inserted into the needle paths 22 and 23 during the manufacturing process of assembling the components of the apparatus 8 prior to sale. An adhesive, such as cyanoacrylate, silicone, or other curable adhesive or sealant may also be utilized to retain the tubular insert 25 in position within the needle paths 27 and 29.

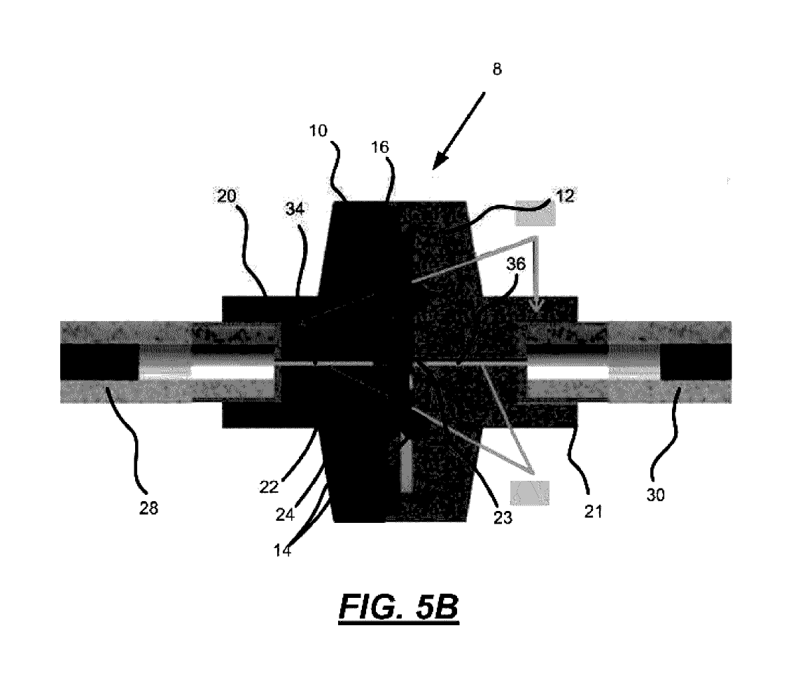

As shown in FIGS. 5A and 5B, the assembled apparatus 8 may accommodate, receive, or have inserted into the apparatus 8 a first syringe 28 having a first needle 34 and a second syringe 30 having a second needle 36 via a first syringe port 20 and a first needle path 22 in the first housing member 10 and a second syringe port 21 and a second needle path 23 in the second housing member 12. Each of the first and second needle paths 22 and 23 may have an inner diameter ranging from about 0.01 to about 10.0 millimeters. Desirably, the diameter of the needle paths 22 and 23 is slightly larger than the outer diameter of the syringe needles inserted into the needle paths 22 and 23, so that there is no leakage of fluid from the needle paths 22 and 23 through the syringe ports 20 and 21. Furthermore, the first housing member 10 may provide mechanical support to the first syringe 28 via a first syringe port 20, and the second housing member 12 may provide mechanical support to a second syringe 30 via a second syringe port 21. The first syringe port 20 and the second syringe port 21 may be dimensioned to precisely and securely accept a specific type or size of syringe, for example, a 1 cc needle-tipped disposable syringe or Hamilton glass reusable syringes, or any other syringe known by those skilled in the art to be suitable for the sizing or extrusion of liposomes, so that when inserted, the first needle 34 may fit into the first needle path 22, and the second needle 36 may fit into the second needle path 23, both needles fitting precisely and securely into the respective needle paths. Likewise, the first syringe 28 may fit into the first syringe port 20, and the second syringe 30 may fit into the second syringe port 21, both syringes fitting precisely and securely into the respective syringe ports, so as to provide a complete leak proof path in fluid communication axially through the assembled apparatus 8, from the first syringe 28 to the second syringe 30. The syringe ports 20 and 21 may have a diameter ranging from about 3 mm to about 30 mm.

In the present embodiment, the first plastic housing member 10 and the second plastic housing member 12 may have a first syringe port 20 and a second syringe port 21 having identical dimensions, as well as a first needle path 22 and a second needle path 23 having identical dimensions. In other embodiments, the first plastic housing member 10 and the second plastic housing member 12 may have a first syringe port 20 and a second syringe port 21 having differing dimensions, and/or a first needle path 22 and a second needle path 23 having differing dimensions.

FIGS. 5A and 5B display an embodiment of the present disclosure featuring an assembled disposable extrusion apparatus 8 having a first syringe 28, which may contain a fluid, and having a first needle 34, being inserted into the first housing member 10. The first syringe 28 may be inserted via the first syringe port 20, whereby the first needle 34 is securely and precisely disposed into the first needle path 22. The apparatus 8 may further have a second syringe 30, which may contain a fluid, and having a second needle 36 being inserted into the second housing member 12. The second syringe 30 may be inserted via the second syringe port 21, whereby the second needle 36 is securely and precisely disposed into the second needle path 23.

A fluid from either the first syringe 28 or the second syringe 30 may thus be transferred or exchanged back and forth across the membrane 16. The fluid may flow through the first needle 34 to or from the first syringe 28 and the through second needle 36 to or from the second syringe 30 via the first needle path 22, the fluid communication path 24, and the second needle path 23 without any leakage of fluid. Accordingly, when using the assembled apparatus 8 for liposome extrusion, there is no leakage of fluid from the first syringe 28 or the second syringe 30, or from the first syringe port 20 or the second syringe port 21, or from the first needle path 22 or the second needle path 23.

The disclosed pre-assembled, single-use extrusion apparatus 8 may be suitable for use when preparing and sizing liposomes or vesicles constructed from materials including, but not limited to, lipids, proteins, cholesterols, cell membranes or fragments thereof, other biomolecules and analogs thereof, polymers, surfactants, and other amphiphilic species.

The embodiment shown in FIGS. 5A and 5B comprises two syringes, however other embodiments may comprise only one syringe and syringe port, or more than two syringes and syringe ports.

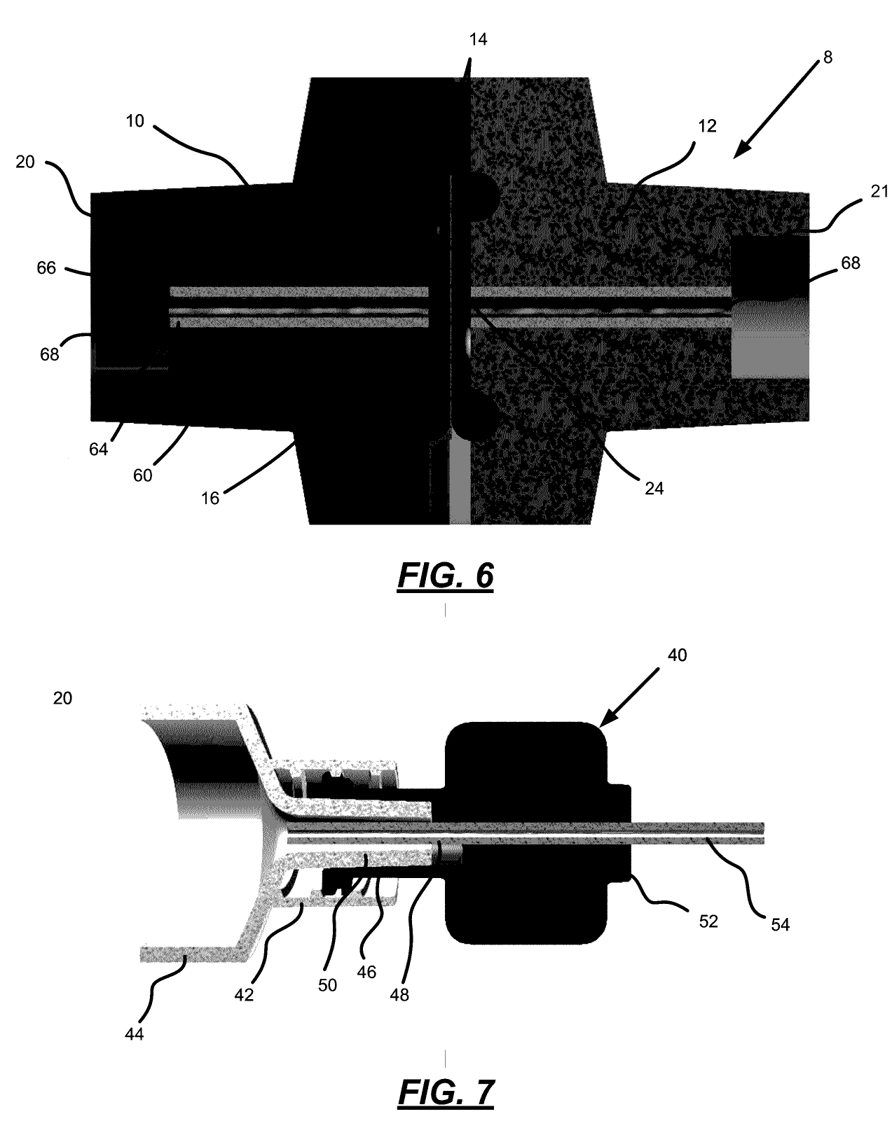

With reference now to FIG. 6, an additional embodiment of the present disclosure may provide a fully assembled, single-use extrusion apparatus 8, wherein the apparatus 8 comprises first and second housing members 10 and 12, enclosing one or more O-rings 14 and at least one extrusion membrane 16, and having first and second syringe ports 20 and 21, as described in detail above. The housing members 10 and 12 may each have a needle path bore 60 extending through the housing members 10 and 12 from the syringe ports 20 and 21 to the low-dead-volume fluid flow path 24. The needle path bore 60 may concentrically enclose an outer tubular insert 64, which may further concentrically enclose an inner tubular insert 66. The outer tubular insert 64 may be may have an outer diameter substantially equal to or just smaller than the needle path bores 60, and an inner diameter substantially equal to or slightly less than the outer diameter of the inner tubular insert 66. The inserts 64 and 66 may be substantially equal in length to the needle path bores 60. The concentric tubular inserts 64 and 66 may aid in maintaining a leak proof fluid communication connection, extending from a needle aperture 68 through the inner tubular insert 64 in the first housing member 10, into the low dead-volume fluid flow path 24, across the membrane 16, and through the inner tubular insert 64 to a needle aperture 68 in the second housing member 12.

The outer tubular insert 64 may be comprised of a flexible elastomeric tubing, such as surgical tubing or silicone tubing. The inner tubular insert 66 may be comprised of a more rigid material, such as polytetrafluoroethylene, non-protein or lipid binding metals such as stainless steel or titanium, or any suitable curable plastic, thermoplastic, polymer, or copolymer that may be non-protein or non-lipid binding, as will be recognized by those of skill in the art. The inner and outer tubular inserts 64 and 66 may be inserted into the needle paths bores 60 during the manufacturing process of assembling the components of the apparatus 8 prior to sale. An adhesive, such as cyanoacrylate, silicone, or other curable adhesive or sealant may also be utilized to retain the tubular inserts 64 and 66 in position within the needle paths bores 60, wherein such adhesive may be disposed around, between, or at one or more ends of at least one of the concentric tubular inserts 64 and 66 and needle path bore 60.

The concentric tubular inserts 64 and 66 may be substantially the same length. The outer tubular insert 64 may be shorter in length that the inner tubular insert 66, and a curable adhesive, including but not limited to, cyanoacrylate, may fill the remainder of the needle path bore 60, and may aid in retaining both tubular inserts 64 and 66. The inserts may comprise one or more sections of tubular material, and in some embodiments, there may be more than two concentric tubular inserts.

In a further embodiment of the present disclosure, the assembled extrusion apparatus 8 may also be used with common luer-lock tipped syringes while retaining a substantially low amount of dead-volume, through the use of an apparatus 8 having at least one of the first syringe port 20 and the second syringe port 21 having at least one of a male luer adapter and a female luer adapter, and further having an embedded needle-like protrusion, as described below.

With reference now to FIG. 7, a low dead-volume luer-needle adapter 40 may be threaded or slipped onto a male or female luer connection 42 of a syringe 44 to create a leak-free fluid path connection. An luer adapted end 46 of the adapter 40 that connects to the luer may include a tubular needle-like protrusion 48 that enters an interior portion 50 of the syringe 44, and may be dimensioned to reduce the inherent dead volume of the syringe 42, as well as provide a leak-proof connection to the syringe. An apparatus adapted end 52 of the adapter 40 may include a tubular needle-like protrusion 54, and both end 52 and protrusion 54 may be dimensioned for leak-proof connection and communication with the fluid path in the single-use, low dead-volume extrusion apparatus described herein. The needle-like protrusions 48 and 54 extending from the two ends 46 and 52 of the adapter 40 may be collinear, and provide fluid flow communication through the adapter 40 from the syringe 44 to the apparatus disclosed herein. The tubular needle-like protrusions 48 and 54 may extend through the adapter 40, and may be comprised of a single tube of stainless steel, titanium, or non-metallic material such as polytetrafluoroethylene, or any other material mentioned above for the construction of the apparatus, as will be appreciated by those of skill in the art. Both the apparatus and adapter 40 may be constructed so as to achieve a practical and cost-effective single-use device that eliminates the need for cleaning and assembly before or after extrusion, thereby significantly reducing both the risk of contamination of the extrudant, and the risk of improper assembly, both of which may have detrimental downstream effects.

In a further embodiment of the present disclosure, the housing members 10 and 12 may have male or female luer connectors incorporated into the syringe ports 20 and 21 so that the apparatus 8 may be used with luer syringes directly, without the need for a separate adapter. In such an embodiment, the syringe ports 20 and 21 may include a needle-like protrusion that extends through the housing into at least one of a needle path or a tubular insert, and which may also protrude from the housing so as to enter the tip of a luer syringe when such a syringe is connected to the syringe ports 20 and 21, thereby maintaining a fluid communication connection between the syringes and the assembled apparatus. In this manner, the dead-volume of a luer syringe may be reduced. The syringe ports 20 and 21 may be further adapted to reduce dead-volume by filling space around the protrusion with a filling agent, such as an adhesive, silicone, plastic, or thermoplastic, curable resin, or other suitable material known to those skilled in the art.

The embodiment disclosed herein comprises an identical first syringe port 20 and second syringe port 21 on either end of the fluid path 24 and the first needle path 22 and the second needle path 23 in the assembled apparatus 8, which allows the user to pass solution back and forth between the first syringe and the second syringe, via the extrusion apparatus 8, through the one or more membranes 16 at least one time, or multiple times, to achieve a desired size of liposomes, vesicles, or other substances, including but not limited to pharmaceuticals, proteins, and other molecules or polymers desired for size selection or extrusion.

Other embodiments of the single-use extrusion apparatus 8 described herein may incorporate multiple extrusion membranes 16 in series, order to achieve an effect equivalent to multiple passes through a single membrane 16 in fewer passes, or possibly even in a single pass. For example, the apparatus may be configured to have about 1 to about 20 extrusion membranes 16. Further embodiments of the present disclosure may be configured to use one or more extrusion membranes 16 of varying sizes in a “step-down” or “step-up” extrusion configuration, wherein the apparatus 8 may enclose or contain one or more membranes 16 whose pore sizes sequentially decrease, or sequentially increase, across the fluid flow path 24.

An example of such a “step-down” extrusion apparatus 8 may have an extrudant passed serially through one or more extrusion membranes 16 having about a 400 nm pore diameter, then one or more membranes 16 having about a 200 nm pore diameter, followed by one or more membranes 16 having about a 50-100 nm pore diameter. Other pore diameters and filtration configurations are feasibly within the context of the single-use, low dead-volume extrusion apparatus 8, as disclosed herein.

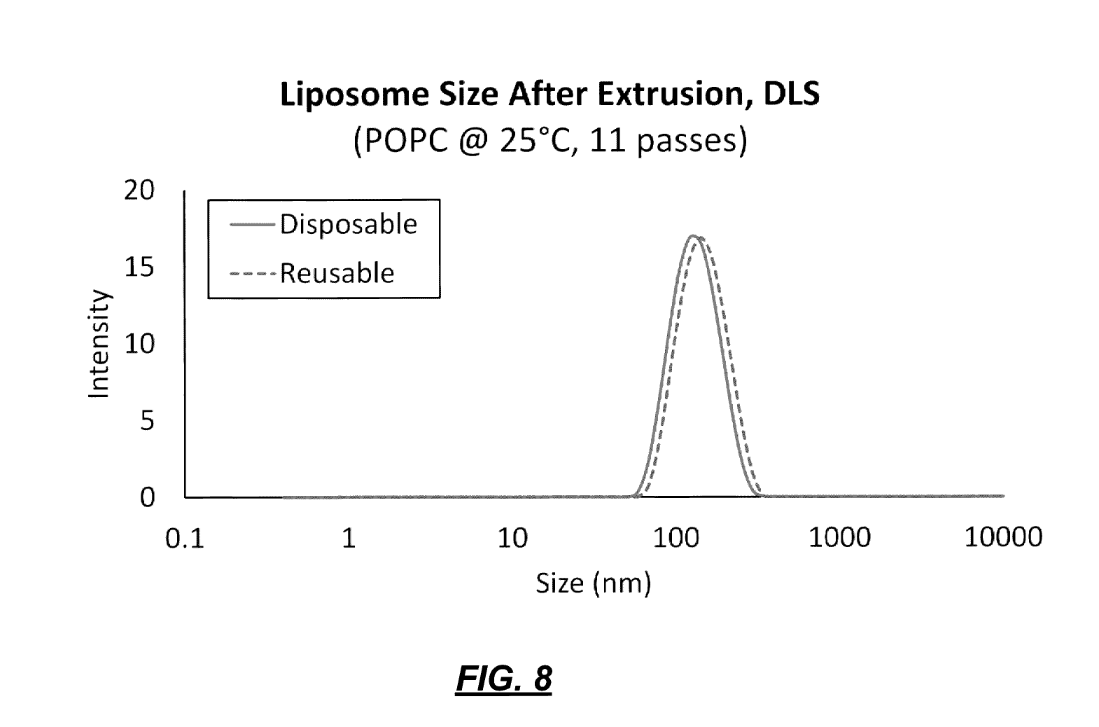

With reference now to FIG. 8, comparative results from extrusions using a disposable apparatus as disclosed herein and a commercially available re-usable extruder are graphed. The size of the liposomes were determined using DLS, dynamic light scattering, a well-established method for measuring the diameter of liposomes, micelles, polymerosomes, and other colloids. Diameter is given in nanometers. The liposomes used for this test were comprised of palmitoyloleoyl phosphatidylcholine (POPC). As shown by the test data, the disposable extrusion apparatus as described herein provides liposomes of comparable size (in this case about 100 nm) to those of currently available re-usable extrusion apparatus. However, extremely advantageously and as previously described herein, the presently disclosed extrusion apparatus is cleaner, faster, and less expensive to use than other re-usable extrusion devices.

The apparatus disclosed herein, and all embodiments thereof, may be sterilized by either the manufacturer or the user. Suitable sterilization techniques may include ethylene oxide sterilization or irradiation, for example gamma ray irradiation. It will be appreciated by those of skill in the art that other sterilization procedures may be utilized, depending on the materials used in construction of the apparatus.

While the disclosure has been described in terms of exemplary embodiments, those skilled in the art will recognize that the disclosure can be practiced with modifications in the spirit and scope of the appended claims. The examples are merely illustrative and are not meant to be an exhaustive list of all possible designs, embodiments, applications or modifications of the disclosure.

The patentees do not intend to dedicate any disclosed embodiments to the public, and to the extent any disclosed modifications or alterations may not literally fall within the scope of the claims, they are considered to be part hereof under the doctrine of equivalents.

What is claimed is:

1. A single-use, low dead-volume extrusion apparatus comprising:
a) a first plastic housing member comprising a first port dimensioned to receive a first needle-tipped syringe, and further comprising a first needle path dimensioned to receive a first needle, wherein the first needle path extends from the first port through the first housing member;
b) a second plastic housing member comprising a second port dimensioned to receive a second needle-tipped syringe, and further comprising a second needle path, dimensioned to receive a second needle, wherein the second needle path extends from the second port through the second housing member and is collinear with the first needle path extending through the first housing member; and
c) a membrane filter disposed between the first and second plastic housing members for interrupting fluid flow from the first needle path to the second needle path,
wherein the first port is in fluid communication with the second port via the first needle path and the second needle path, and wherein the first housing member and the second housing member are fixedly attached to one another with the member filter disposed therein;
and wherein a total dead-volume of the apparatus, not including the first and second needle paths, is less than or equal to about 50 microliters.

2. The apparatus of claim 1, wherein the first housing member and the second housing member further comprise one or more elastomeric seals or o-rings disposed in the first and/or second housing members and circumscribing the membrane filter.

3. The apparatus of claim 1, wherein the one or more extrusion membranes have a pore size ranging from about 10 nm to less than about 1000 nm.

4. The apparatus of claim 1, wherein the apparatus contains two or more extrusion membranes each having substantially the same pore size.

5. The apparatus of claim 1, wherein the apparatus contains two or more extrusion membranes each having substantially different pore sizes.

6. The apparatus of claim 1, wherein the apparatus contains two or more extrusion membranes each having substantially different pore sizes and wherein the extrusion membranes are arranged in order of increasing pore size or decreasing pore size.

7. The apparatus of claim 1, wherein the extrusion membrane comprises a track-etched polycarbonate membrane having a pore size ranging from 50 to about 500 nm.

8. The apparatus of claim 1 wherein the first port further comprises a luer-lock adapter having low dead-volume, and a needle connected to the luer lock adapter, wherein the needle is in fluid communication with the apparatus.

9. A method for extruding liposomes via a single-use, sterile extrusion apparatus, comprising:
inserting a first needle-tipped syringe into a first plastic housing member, wherein the first plastic housing member comprises a first port, wherein the first port is dimensioned to receive the first needle-tipped syringe, and wherein the first housing member further comprises a first needle path, dimensioned to receive a first needle, wherein the first needle path extends from the first port through the first housing member, and wherein the first needle is in fluid communication with the first needle-tipped syringe;
b) inserting a second needle-tipped syringe into a second plastic housing member, wherein the second plastic housing member comprises a second port, wherein the second port is dimensioned to receive the second needle-tipped syringe, and wherein the second housing member further comprises a second needle path dimensioned to receive a second needle, wherein the second needle path extends from the second port through the first housing member and is collinear with the first needle path extending through the first housing member, and wherein the second needle is in fluid communication with the second needle-tipped syringe; wherein the extrusion apparatus comprises one or more extrusion membrane disposed between the first and second housing members for interrupting fluid flow from the first needle path to the second needle path;
c) displacing a fluid comprising at least one liposome from the first needle-tipped syringe to the second needle-tipped syringe, through the extrusion apparatus so that the fluid flows through the one or more extrusion membranes, and at least one liposome in the fluid passes through the one or more extrusion membranes wherein the one or more membranes reduce the size or lamellarity of the liposome, and wherein there is no leakage of fluid;
d) and displacing the fluid and at least one liposome from the second needle-tipped syringe to the first needle tipped syringe, through the extrusion apparatus via the extrusion membrane, without leakage of fluid.

10. The method of claim 9, wherein the first housing member and the second housing member are fixedly attached to one another, and wherein the first port is in fluid communication with the second port via the first needle path and the second needle path.

11. The method of claim 9, wherein at least one of the first needle-tipped syringe and second needle-tipped syringe contains a fluid.

12. The method of claim 9, wherein the first needle-tipped syringe and the second needle-tipped syringe are in fluid flow communication with the apparatus.

13. The method of claim 9, wherein the dead volume of the extrusion apparatus, not including the dead volume of the first and second needle-tipped syringes is less than or equal to about 50 microliters.

14. The method of claim 9, wherein the first housing member and the second housing member further enclose one or more o-ring seals.

15. The method of claim 9, wherein the fluid comprises one or more liposomes.

16. The method of claim 9, wherein the one or more extrusion membrane have a pore size ranging from about 10 nm to less than about 1000 nm.

17. The method of claim 9, wherein the apparatus comprises two or more extrusion membranes having substantially the same pore size.

18. The method of claim 9, wherein the apparatus comprises two or more extrusion membranes having substantially different pore sizes.

19. The method of claim 9, wherein the apparatus comprises two or more extrusion membranes having substantially different pore sizes and wherein the extrusion membranes are arranged in order of increasing pore size or decreasing pore size.

20. The method of claim 9, wherein the extrusion membrane comprises a track-etched polycarbonate membrane having a pore size ranging from 50 to about 500 nm.

21. The method of claim 9, wherein the fluid comprises one or more liposomes, and wherein the one or more liposomes comprises at least one of lipids, proteins, cholesterols, cell membranes or fragments thereof, other biomolecules and analogs thereof, polymers, surfactants, and other amphiphilic species.
